(19) United States
(12) Patent Application Publication
(10) Pub. No.: US 2025/0114038 A1
(43) Pub. Date: Apr. 10, 2025

(54) SMART GARMENT AND SYSTEM FOR IDENTIFYING BREATHING EVENTS

(71) Applicant: W. L. Gore & Associates, Inc., Newark, DE (US)

(72) Inventors: Paul Knight, Spokane, WA (US); Cameron A. Mertz, Bridgewater, NJ (US); Kelsey A. Tacca, Columbus, OH (US)

(21) Appl. No.: 18/910,378

(22) Filed: Oct. 9, 2024

Related U.S. Application Data
(60) Provisional application No. 63/543,430, filed on Oct. 10, 2023.

(51) Int. Cl. A61B 5/00; A41D 1/00; A61B 5/08

(52) U.S. Cl. CPC A61B 5/6804; A41D 1/002; A61B 5/0826

(57) ABSTRACT

The present disclosure relates to systems and methods for identifying breathing events of a subject using garments with sensors and associated computer systems. More specifically, the disclosure relates to garments and computer systems for identifying individual elements of a breathing event, also called breathing event features and methods for determining the breathing events of users wearing the garments.

### CROSS-REFERENCE TO RELATED APPLICATION

This application claims the benefit of Provisional Application No. 63/543,430, filed Oct. 10, 2023, which is incorporated herein by reference in its entirety for all purposes.

### FIELD

The present disclosure relates generally to garments with sensors and associated computer systems. More specifically, the disclosure relates to garments and computer systems for identifying breathing events of users wearing the garments.

### BACKGROUND

Wearable electronics and smart garments or apparel are becoming increasingly popular. These smart garments, which include sensors and other electronic components, can be used to collect a wide range of information about the user wearing the garment. Examples of such information include physiologic information, such as the pulse rate and oxygen saturation of a wearer, and ergonomic or movement information. There remains, however, a continuing need for improved smart garments and associated systems for processing data collected by the smart garments.

### SUMMARY

Smart garments and associated computer systems and methods in accordance with the disclosed examples may provide a number of advantages. For example, they are capable of efficiently and accurately providing useful insights into activities and physiologic conditions of a subject.

One example is a method for operating a computing system including one or more processors to identify breathing events of a subject. Embodiments may comprise receiving, by the one or more processors, data from a plurality of sensors, including a plurality of motion sensors, mounted to an article positioned on an upper body of the subject, wherein the data includes data associated with movement of an upper body of the subject; processing the data, by the one or more processors, to identify a breathing event of the subject, wherein the identified breathing event is at least one of a normal intensity breathing event, a medium intensity breathing event, a high intensity breathing event, a low intensity talking event, a normal intensity talking event, a medium intensity talking event, a high intensity talking event, a cough event, a sneeze event, a choke event, a scream event, and an apnea event; wherein at least two of the plurality of motion sensors are mounted to the article at positions corresponding to a midline of an anterior portion of the subject; and wherein at least a first of the plurality of motion sensors is mounted to the article at a position corresponding to a position proximate to and above an umbilicus of the subject, and at least a second of the plurality of motion sensors is mounted to the article at a position corresponding to a position proximate to and below the umbilicus of the subject.

In some embodiments of the method, processing the data to identify a breathing event comprises comparing the data to stored breathing event data. In embodiments, the method may further comprise receiving and storing the breathing event data. In embodiments, the stored breathing event data may be representative of one or more upper body poses. In embodiments, the stored breathing event data may include data received during a static upper body pose. In embodiments, the stored breathing event data may include data received during a dynamic upper body pose.

In any or all of these embodiments, processing the data may comprises processing the data by a trained model. In some embodiments, processing the data comprises processing the data by a trained artificial neural network. Any or all of these embodiments may further comprise training the model.

In any or all of these embodiments, processing the data may comprise: converting at least portions of the data into context data associated with human-relatable values; and processing the context data.

In any or all of these embodiments, receiving the data may include receiving data from each of at least two zones of the article; and processing the data includes processing the data from the at least two zones.

In any or all of these embodiments, receiving the data may comprise receiving the data from the plurality of sensors mounted to a shirt.

In any or all of these embodiments, processing the data may include identifying features associated with movement of one or more of the subject's chest, upper abdomen, lower abdomen, left arm or right arm.

In any or all of these embodiments, processing the data may include identifying breathing event features.

In any or all of these embodiments, processing the data to identify a breathing event may include determining an angle of the upper body of the subject based upon data from sensors including the at least two of the plurality of motion sensors mounted at positions corresponding to the midline of the anterior portion of the subject, the at least first of the plurality of motion sensors mounted at the position proximate to and above the umbilicus, and the at least second of the plurality of motion sensors mounted at the position proximate to and below the umbilicus.

Another example is a system for identifying breathing events of a subject. Embodiments of the system may comprise: an article configured to be positioned on an upper body of the subject, the article including a plurality of sensors, including a plurality of motion sensors, for sensing subject data; and a computer system including one or more processors, wherein the computer system is configured to receive the subject data and to identify a breathing event of the subject, wherein the breathing event is at least one of a normal intensity breathing event, a medium intensity breathing event, a high intensity breathing event, a cough event, a sneeze event, a choke event, a low intensity talking event, a normal intensity talking event, a medium intensity talking event, a high intensity talking event, a scream event, and an apnea event; wherein at least two of the plurality of motion sensors are located on the article at positions corresponding to a midline of an anterior portion of the subject; and wherein at least a first of the plurality of motion sensors is located on the article at a position corresponding to a position proximate to and above an umbilicus of the subject, and at least a second of the plurality of motion sensors is located on the article at a position corresponding to a position proximate to and below the umbilicus of the subject.

In some embodiments of the system, the article further includes one or more sensors from a group including a PPG sensor, an implantable sensor, a light sensor, a heart sensor, a location sensor, a bioimpedance sensor, an EMG sensor, a GPS sensor, a microphone sensor, an environmental sensor, a bend sensor, and a stretch sensor.

In some embodiments of the system, the article further comprises a processor configured to be removably mounted to the article and coupled to one or more of the plurality of motion sensors. In some embodiments of the system, the article further comprises electrical conductors coupling one or more the plurality of motion sensors to one or more others of the plurality of motion sensors and/or to the processor.

In some embodiments of the system, the article further comprises memory for storing the sensor data.

In some embodiments of the system, the article further comprises a wireless transmitter for transmitting the sensor data.

In some embodiments of the system, the plurality of motion sensors comprises one or more motion sensors in each of at least two zones. In some embodiments of the system, the article comprises a shirt and the at least two zones include zones from a group including a chest zone, an upper abdomen zone, a lower abdomen zone, a left arm zone and a right arm zone.

In some embodiments of the system, the article comprises a garment.

In some embodiments of the system, the article comprises a shirt.

In some embodiments of the system, the computer system identifies the breathing event at least in part by determining an angle of the upper body of the subject based upon subject data from sensors including the at least two of the plurality of motion sensors mounted at positions corresponding to the midline of the anterior portion of the subject, the at least first of the plurality of motion sensors mounted at the position proximate to and above the umbilicus, and the at least second of the plurality of motion sensors mounted at the position proximate to and below the umbilicus.

In some embodiments of the system, the computer system identifies the breathing event at least in part by comparing the subject data to stored breathing event data. In some embodiments of the system, the computer system receives and stores the breathing event data. In some embodiments of the system, the stored breathing event data is representative of one or more upper body poses. In some embodiments of the system, the stored breathing event data includes data received during a static upper body pose. In some embodiments of the system, the stored breathing event data includes data received during a dynamic upper body pose.

In some embodiments of the system, the computer system identifies the breathing event at least in part by processing the subject data by a trained model. In some embodiments, the trained model is a trained artificial neural network. In some embodiments, the computer system is configured to train the model.

In some embodiments of the system, the computer system identifies the breathing event at least in part by: converting at least portions of the subject data into context data associated with human-relatable values; and processing the context data.

In some embodiments of the system, the computer system identifies the breathing event at least in part based upon subject data from each of at least two zones of the article; and processing the data from the at least two zones.

In some embodiments of the system, the computer system identifies the breathing event at least in part by identifying features associated with movement of one or more of the subject's chest, upper abdomen, lower abdomen, left arm or right arm.

In some embodiments of the system, the computer system identifies the breathing event at least in part by identifying breathing event features.

Another example is an article. Embodiments of the article may comprise: a shirt configured to be worn on an upper body of a subject; a plurality of sensors, including a plurality of motion sensors, mounted to the shirt for providing subject data, wherein the plurality of motion sensors are configured and arranged to provide subject data characteristic of sufficient features associated with the upper body of the subject, including at least one feature associated with movement of the upper body of the subject, to determine a subject breathing event, wherein the breathing event is at least one of a normal intensity breathing event, a medium intensity breathing event, a high intensity breathing event, a cough event, a sneeze event, a choke event, a low intensity talking event, normal intensity talking event, a medium intensity talking event, a high intensity talking event, a scream event, and an apnea event; a data transfer structure on the shirt coupled to the plurality of sensors and configured to facilitate transferring the subject data off of the shirt; wherein at least two of the plurality of motion sensors are located on the shirt at positions corresponding to a midline of an anterior portion of the subject; and wherein at least a first of the plurality of motion sensors is located on the shirt at a position corresponding to a position proximate to and above an umbilicus of the subject, and at least a second of the plurality of motion sensors is located on the shirt at a position corresponding to a position proximate to and below the umbilicus of the subject.

In some embodiments of the article, the plurality of motion sensors includes one or more sensors on at least two zones of the shirt.

In some embodiments of the article: the shirt includes a chest zone, an upper abdomen zone, a lower abdomen zone, a left arm zone and a right arm zone, and the plurality of motion sensors includes one or more motion sensors on each of the chest zone, upper abdomen zone, lower abdomen zone, left arm zone and right arm zone.

In some embodiments of the article, the data transfer structure comprises a transmitter.

In some embodiments of the article, the data transfer structure comprises memory for storing the subject data.

Some embodiments of the article further comprise one or more sensors from a group including a PPG sensor, an implantable sensor, a light sensor, a heart sensor, a location sensor, a bioimpedance sensor, an EMG sensor, a GPS sensor, a microphone sensor, an environmental sensor, a bend sensor, and a stretch sensor.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a diagrammatic illustration of a system including a smart garment, shown for example as a shirt, and computer system, in accordance with embodiments.

FIG. 2A is a detailed diagrammatic illustration of a front side of a smart garment, shown for example as a shirt, on a subject, in accordance with embodiments.

FIG. 2B is a detailed diagrammatic illustration of a back side of the shirt shown in FIG. 2A (but not on a subject), in accordance with embodiments.

FIG. 3A-3C illustrate exemplary motion data in the form of acceleration information provided by motion sensors at several locations corresponding to a torso zone of a subject, in accordance with embodiments.

FIG. 4A-4B illustrate exemplary motion data in the form of acceleration information provided by motion sensors at locations corresponding to arm zones of a subject, in accordance with embodiments.

FIG. 5 illustrates exemplary sound data provided by a sound sensor such as a microphone, and diagrammatic illustrations of a corresponding breathing event of a subject, in accordance with embodiments.

FIG. 6 illustrates exemplary physiologic data in the form or heart rate and oxygen saturation of a subject provided by sensors, and corresponding sound data such as that shown in FIG. 5, in accordance with embodiments.

FIG. 7 illustrates exemplary location data of a subject provided by global positioning system (GPS) sensors, in accordance with embodiments.

FIGS. 8A-8D illustrate exemplary environmental data in form of relative humidity, temperature, gas resistance and atmospheric pressure provided by sensors, in accordance with embodiments.

FIG. 9 is a diagrammatic illustration of a method for training a model to identify breathing events, in accordance with embodiments.

FIG. 10 is a diagrammatic illustration of a subject during a breathing event, in accordance with embodiments.

FIG. 11 is a diagrammatic illustration of a subject during a breathing event, in accordance with embodiments.

FIG. 12 is a diagrammatic illustration of a video system for obtaining training data, in accordance with embodiments.

FIGS. 13A and 13B are diagrammatic illustrations of motion sensors on the torso of a subject during breathing events.

FIG. 14 is a diagrammatic illustration of a method for using a model to identify breathing events, in accordance with embodiments.

FIG. 15 is a detailed diagrammatic illustration of a computer system such as that shown in FIG. 1, in accordance with embodiments.

### DETAILED DESCRIPTION

The disclosures of all cited patent and non-patent literature are incorporated herein by reference in their entirety.

As used herein, the term “embodiment” or “disclosure” is not meant to be limiting, but applies generally to any of the embodiments defined in the claims or described herein. These terms are used interchangeably herein.

Unless otherwise disclosed, the terms “a” and “an” as used herein are intended to encompass one or more (i.e., at least one) of a referenced feature.

The features and advantages of the present disclosure will be more readily understood, by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination. In addition, references to the singular may also include the plural (for example, “a” and “an” may refer to one or more) unless the context specifically states otherwise.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

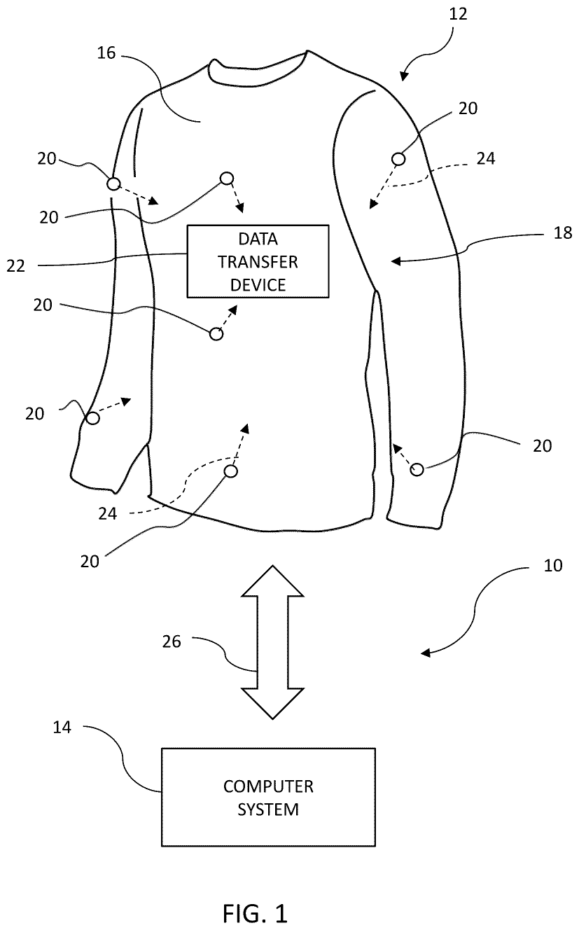

FIG. 1 is a diagrammatic illustration of a system 10 for identifying and classifying breathing events of a subject, in accordance with embodiments. As shown, system 10 includes a smart garment 12 coupled to a computer system 14. The illustrated embodiments of the smart garment 12 include an article, shown for example as a shirt 16 that is configured to be worn by or otherwise positioned on the body of a person or other subject (not shown in FIG. 1), and a plurality of sensors 18 mounted or otherwise coupled to the shirt or the upper body of the subject. As described in greater detail below, the sensors 18 generate sensor data associated with the subject 30 wearing the shirt 16. As shown for example in FIG. 1, sensors 18 include a plurality of motion sensors 20 that provide subject data, for example, motion data, such as location and/or acceleration data, to the computer system 14. In the illustrated embodiments, sensor data from the sensors 18, including the motion data from motion sensors 20, is coupled to a data transfer device 22 on the shirt 16 via transmission channels 24. As described in greater detail below, the transmission channels 24 may be wired or wireless channels. Data transfer device 22, which as described in greater detail below may be a wired or wireless data transmission device, transfers the sensor data, including the motion data, to the computer system 14 via a network 26. In some embodiments, electrical conductors can couple one or more of the plurality of sensors to one or more others of the plurality of motion sensors and/or to the processor.

Computer system 14 processes the sensor data, including the motion data, to identify and classify physical and/or physiologic activity of the subject wearing the smart garment 12. Embodiments of the computer system 12 can identify and classify breathing events of the subject. For example, identified breathing events can be classified as one or more of (1) a normal intensity breathing event, (2) a medium intensity breathing event, (3) a cough event, (4) a sneeze event, (5) a choke event, (6) a low intensity talking event, (7) a normal intensity talking event, (8) a medium intensity talking event, (9) a high intensity talking event, (10) a scream event and (11) an apnea event. As described in greater detail below, in some embodiments, the computer system 14 processes the sensor data using trained machine learning models to identify and classify the breathing events or other physical or physiological activities of the subject. In other embodiments, the computer system 14 compares the sensor data to stored breathing event data representative of the breathing events to identify and classify breathing events. Sensor data provided by the smart garment 12 can also be used by the computer system 14 for certain set-up operations, such as to generate calibration and other data used to train the models and/or to generate the stored breathing event data used to identify and classify the breathing events. Calibration data and trained models of these types effectively provide digital representations or models of the associated breathing events or other physical or physiological activities of the subject.

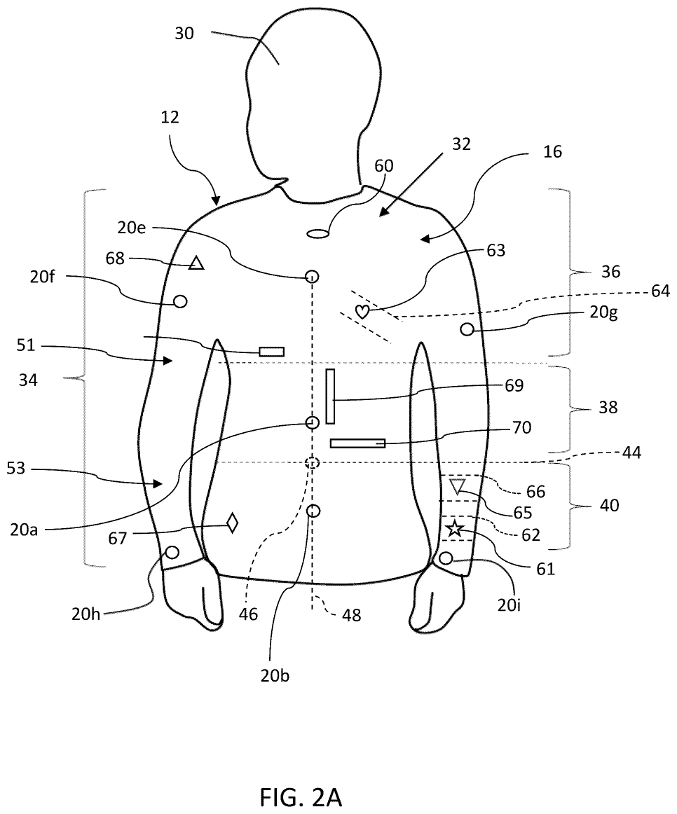

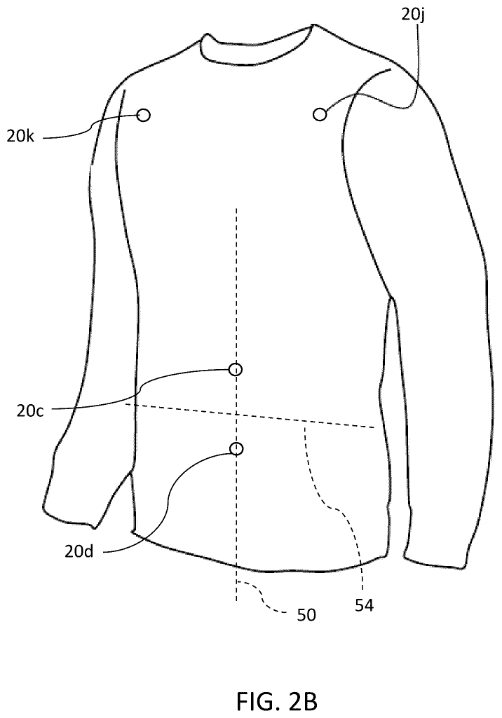

FIG. 2A is a diagrammatic illustration of a front side of a smart garment 12 in accordance with embodiments, where the smart garment is worn by or otherwise positioned on a subject 30. FIG. 2B is a diagrammatic illustration of a back side of the smart garment 12 shown in FIG. 2A. As shown, the smart garment 12 includes a shirt 16 and has portions located adjacent to a number of different portions or zones of the upper body of the subject 30. The torso 32 of the subject 30 includes a torso zone 34 that generally includes a chest zone 36, an upper abdomen zone 38 and a lower abdomen zone 40. A boundary between the upper abdomen zone 38 and the lower abdomen zone 40 may be defined generally by a transverse axis 44 that extend through the umbilicus 46 of the subject 30. The front or anterior side of the torso 32 of the subject 30 is defined by a longitudinal midline 48 that extends generally between inferior anterior portions of the subject. Similarly, the back or posterior side of the torso 32 is defined by a longitudinal midline 50, and by a transverse axis 54 that corresponds generally to the location of the transverse axis 44 on the anterior side of the subject 30. The illustrated embodiments of the shirt 16 also include left and right arm portions that generally include an upper arm zone 51 and a lower arm zone 53, where the upper and lower arm zones are generally defined by the portions of the arms above and below the elbows of the subject 30.

Although shown as a long-sleeved shirt 16 in the illustrated embodiments, other embodiments of the article or smart garment 12 take other forms. Portions of the shirt 16 configured to be located adjacent the zones such as 34, 36, 38, 51 and 51 are also referred to as corresponding zones in this disclosure. For example, other embodiments include short sleeve and sleeveless shirts. Additionally, or alternatively, embodiments of the smart garment 12 include one or more straps or bands configured to be mounted to or otherwise positioned on the subject 30 (and optionally incorporated into the smart garment) for purposes of effectively attaching the sensors 18 to appropriate locations on the upper body of the subject.

The sensors 18 include a first motion sensor 20a at a location corresponding to a position proximate to and above the umbilicus 46 of the subject 30 (e.g., within the upper abdomen zone 38), and a second motion sensor 20b at a location corresponding to a position proximate to and below the umbilicus of the subject (e.g., within the lower abdomen zone 40). In the illustrated embodiments, the first and second motion sensors 20a and 20b are both located at positions that correspond to the midline 48 on the anterior side of the subject 30. The first and second motion sensors 20a and 20b are therefore two motion sensors mounted to the shirt 16 at positions corresponding to the midline 48 of the anterior portion of the subject 30. It has been found that the position of sensors 20a and 20b above and below the umbilicus of the subject produces data that can be used to at least characterize shapes of the abdomen, relative motions of the abdomen, and angles of the abdomen, all of which can contribute to the identification of a type of breathing event.

Embodiments of the smart garment 12, such as for example the embodiments shown in FIG. 2B, may also include a motion sensor 20c at a location on the backside of the shirt 16 that corresponds to a position proximate to and above the transverse axis 54, and a motion sensor 20d at a location on the backside of the shirt proximate to and below the transverse axis 54. In the embodiments shown for example in FIG. 2B, the motion sensors 20c and 20d are mounted at positions corresponding to the midline 50 on the posterior side of the subject 30. In embodiments of these types, the motion sensors 20c and 20d on the back side of the shirt 16 are effectively mounted to locations corresponding to positions proximate to and above and below the umbilicus 46, and are two motion sensors mounted at positions corresponding to the midline 50. At least portions of the motion data provided by motion sensors 20c and 20d may correspond to and/or be used alternatively, or as a surrogate, for the motion data provided by motion sensors 20a and 20b.

Embodiments of the smart garment 12 may include additional sensors 18. The embodiments shown in FIGS. 2A, for example, include a motion sensor 20e located at a position corresponding to the chest zone 36 (and corresponding to the midline 48 in the illustrated embodiments), motion sensors 20f and 20g corresponding to the upper arm zones 51 the right and left upper arms, respectively, and motion sensors 20h and 20i corresponding to the lower arm zones 53 of the right and left arms, respectively (e.g., at positions proximate to the wrists of the subject). The embodiments shown in FIG. 2B include motion sensors 20j and 20k at locations corresponding to the right and left shoulders of the subject 30. Other embodiments of the smart garment 12 include more or fewer motion sensors, and/or motion sensors at different locations on the shirt 16 to provide motion data associated with different portions of the torso 32, arms and/or other parts of the body of the subject 30. In general, the motion sensors 20a-20k are mounted to locations or positions on the shirt 16 corresponding to the locations on the body of the subject 30 where measured motion data can provide information useful in the identification and classification of breathing event features and breathing events.

Motion sensors such as 20a-20k (collectively and along with any other motion sensors on embodiments of the smart garment 12 referred to by reference no. 20) can, for example, be commercially available or otherwise known devices such as Inertial Measurement Units (IMUs). Devices of these types may have an accelerometer, a gyroscope and/or a magnetometer. Sensor data in the form of motion data or information provided by devices of these types includes acceleration data, x-axis, y-axis and/or z-axis movement data, and direction or heading data. Motion sensors 20 of these types may also be used to determine relative angles between portions of the body of the subject 30, such as for example joint angles. In embodiments, motion sensors 20 can provide information at a rate at least as great as 10 Hz, although they may transmit the motion data (e.g., to the computer system 14) at higher or lower rates in embodiments. As used herein, the term “motion sensor” means a sensor comprising at least a device capable of measuring 3-dimensional movement (i.e., an IMU). The motion sensor may also be capable of measuring any of the other environmental, physical, and physiological parameters that are mentioned below. Any of the data captured by the sensors is known as subject data and can be data related to the movement of the subject and any environmental, physical, and physiological parameters of or in proximity to the subject.

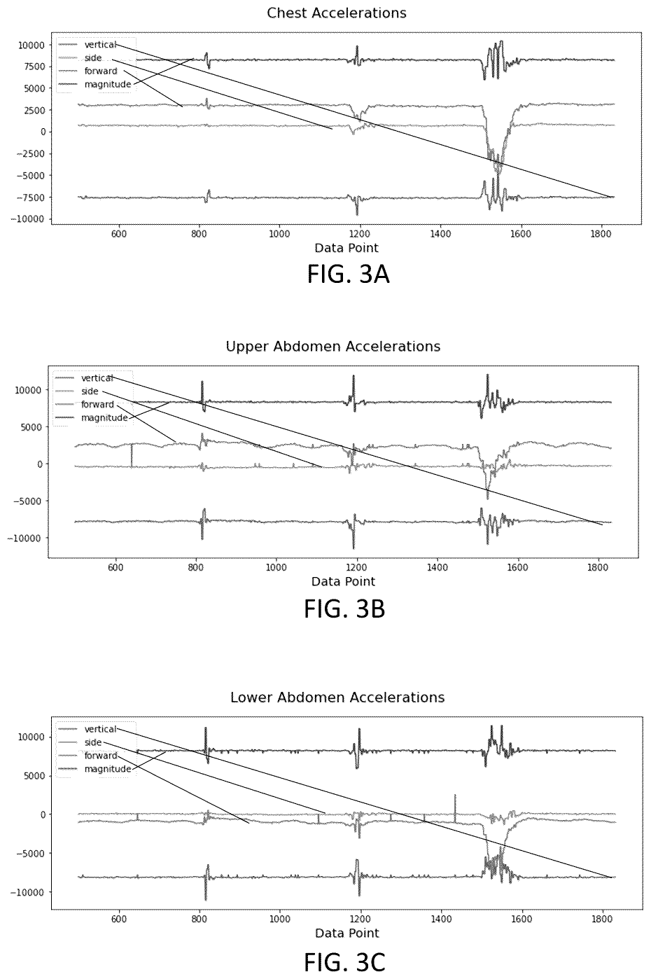

FIG. 3A-3C illustrate exemplary motion data in the form of acceleration information provided by motion sensors 20 at locations corresponding to a torso zone 34 of a subject 30. FIG. 3A, for example, is an illustration of exemplary motion data in the form of acceleration information provided by a motion sensor such as 20e at a position corresponding to the chest zone 36 of a subject 30. Motion data for accelerations in vertical, side, forward and overall magnitude are shown. Overall magnitude can, for example, be computed as the square root of the sum of the squares of the accelerations in the vertical, side and forward directions. FIG. 3B is an illustration of exemplary motion data in the form of acceleration information provided by a motion sensor such as 20a at a position corresponding to the upper abdomen zone 38 of a subject 30. Motion data for accelerations in vertical, side, forward and overall magnitude are shown. FIG. 3C is an illustration of exemplary motion data in the form of acceleration information provided by a motion sensor such as 20b at a position corresponding to the lower abdomen zone 40 of a subject 30. Motion data for accelerations in vertical, side, forward and overall magnitude are shown.

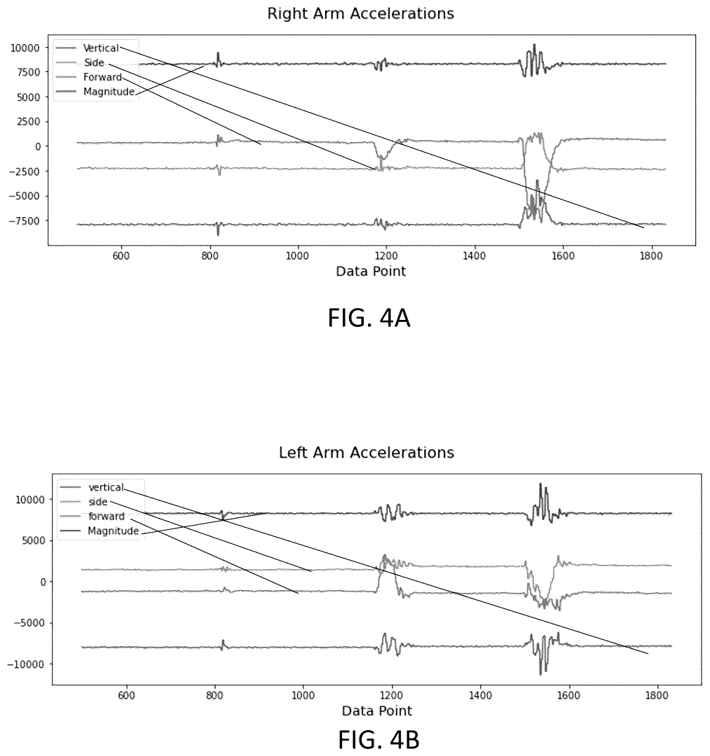

FIGS. 4A-4B illustrate exemplary motion data in the form of acceleration information provided by motion sensors such as 20f-20i at locations corresponding to left and right arm zones 51 and/or 53 of the subject 30, for example during a cough event. FIG. 4A, for example, is an illustration of exemplary motion data in the form of acceleration information provided by a motion sensor such as 20h at a position corresponding to the lower right arm of a subject 30. Motion data for accelerations in vertical, side, forward and overall magnitude are shown. FIG. 4B is an illustration of exemplary motion data in the form of acceleration information provided by a motion sensor such as 20i at a position corresponding to the lower left arm of a subject 30. Motion data for accelerations in vertical, side, forward and overall magnitude are shown.

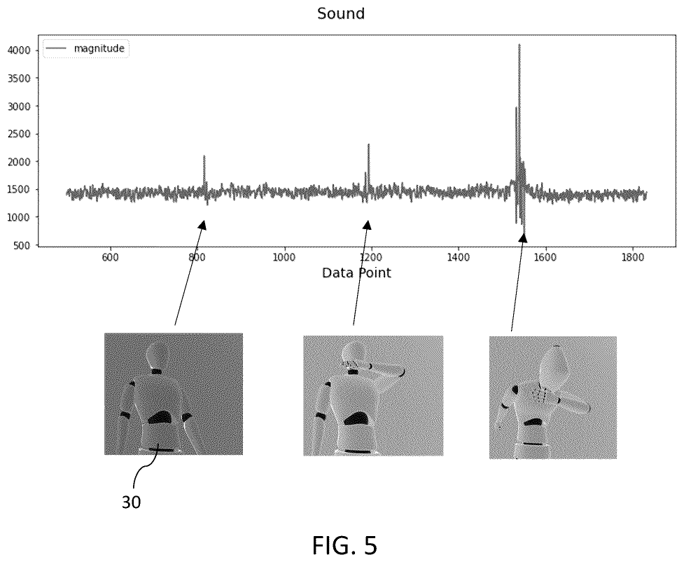

Embodiments of the smart garment 12 may also include other sensors 18. The other sensors 18 can be one or more of a photoplethysmography (PPG) sensor, an implantable sensor, a light sensor, a heart sensor, a location sensor, a bioimpedance sensor, an EMG sensor, a GPS sensor, a microphone or sound sensor, an environmental sensor, a bend sensor, and a stretch sensor. The embodiments shown in FIG. 2A, for example, includes a sound sensor in the form of a microphone 60. Microphone 60 may be any commercially available or otherwise known device used to detect sound, for example either full spectrum or specific frequencies of sound. In embodiments, for example, microphone 60 may measure sound decibels, and at a rate sufficiently high to provide information sufficient to enable the identification of events such as breathing events. In embodiments, the sound information collected or used from the microphone is not used to detect voice communications. For example, the collected frequencies of the sound may be low enough to effectively prevent the detection of voice communications. For example, microphone 60 may be configured to detect sound at a rate at or below 10 Hz. Higher or lower rates of detection can be used in other embodiments. FIG. 5 illustrates exemplary sensor data in the form of sound data provided by a microphone 60, during a series of three cough events. The magnitude of the sound detected by the microphone 60 increases sequentially for each of the cough events in the example shown in FIG. 5. Exemplary positions of certain body portions of the subject 30 during each of the three cough events, including the torso zone 34, chest zone 36, upper abdomen zone 38, lower abdomen zone 40 and upper and lower arm zones 51, 53, are also shown in FIG. 5. In some embodiments, the microphone is a directional microphone that is oriented in such a way that the microphone is configured to capture the sounds of the breathing event, but minimizes the amount of ambient environmental sounds that might be captured, for example, the voice of the subject or voices of those around the subject of the garment.

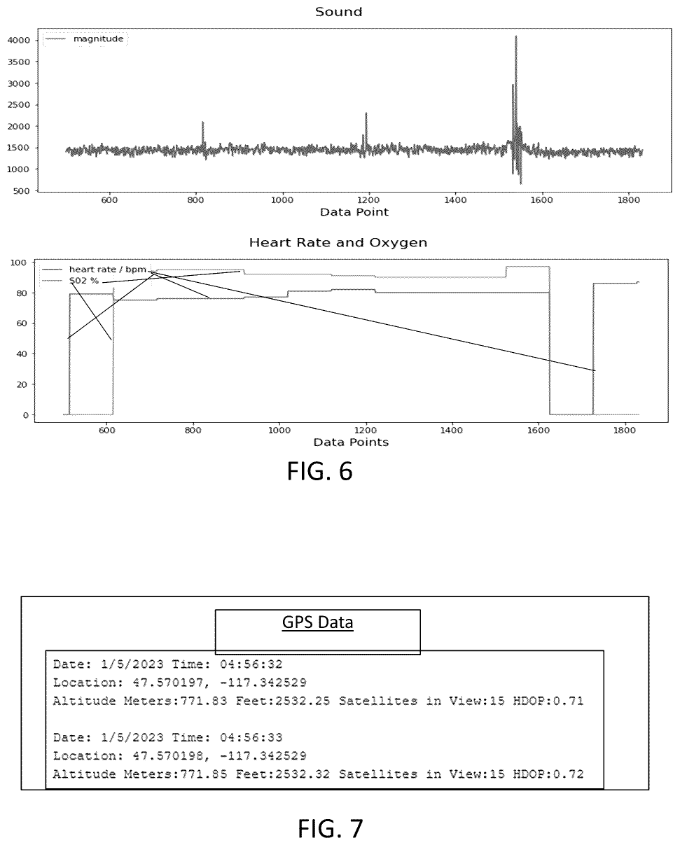

A light sensor 61 is also included in the embodiments shown in FIG. 2A. Light sensor 61 may direct light (e.g., from one or more LEDs) onto the body of the subject 30, and detect or sense (e.g., by one or more photodetectors), reflected or otherwise returned light representative of how the emitted light is absorbed and/or reflected. Light sensor 61 may be any commercially available or otherwise known device that provides information about the subject 30, such as for example pulse oximetry or photoplethysmography (PPG) devices. Light sensors 61 of these types can, for example, provide sensor data or information representative of physiologic characteristics such as blood, heart rate, blood oxygenation and blood pressures. FIG. 6 illustrates exemplary sensor data in the form of heart rate and blood oxygenation levels provided by a light sensor 61. Exemplary sound data provided by a microphone 60 (e.g., representative of cough events) at times corresponding to the sensor data provided by the light sensor 61 is also shown in FIG. 6. In the illustrated example, heart rate of the subject 30 increased with coughing, while blood oxygenation levels remained relatively steady. Some limited loss of signal level provided by the light sensor 61 with body movement of the subject 30 is represented by FIG. 6.

Embodiments may include a band 62 to enhance or otherwise optimize the pressure by which the light sensor 61 is urged into functional engagement with the body of the subject 30. For example, too much pressure may be uncomfortable, and too little pressure may increase risk of inaccurate data sensing. Band 62 may be structure, such as a compression band, that is effectively incorporated into the shirt 16 (and optionally removable), or a structure separate from the shirt. Pressure exerted by any band 62 may be a function of the type of smart garment 12 to which the light sensor 61 is mounted, and the application. The form factor of the smart garment 12 may provide the ability for optimized bands 62 that provide a comfortable feel and fit. The band 62 may, for example, be sewn or otherwise attached to the shirt 16. In some embodiments the sleeve cuffs of the shirt 16 may be configured to function as the band 62. In embodiments, for example, the band 62 may have a material stiffness in stretching that is greater than the material of the shirt 16. The band 62 may apply optimal pressure on the sensor 61 toward the skin of the subject 30, without requiring other portions of the shirt 16 to be uncomfortably tight. Although light sensor 61 is shown for purposes of example at a position corresponding to a wrist of the subject 30, it may be located at other positions, such as for example a shoulder of the subject, in other embodiments (e.g., short sleeve shirt embodiments).

A heart sensor 63 is also included in the embodiments shown in FIG. 2A. Heart sensor 63 may be any commercially available or otherwise known device that measures parameters of the heart of subject 30. In embodiments, for example, heart sensor 63 may be an electrocardiogram (ECG or EKG) sensor that measures the electrical performance or characteristics of the heart. Although shown on the front side of the subject 30, heart sensor 63, which may include wet or dry contact electrodes (not shown), may alternatively or additionally be located on the back of the subject. The heart sensor 63 may be used with a band 64, which may for example have features and functionality of the band 62 of the light sensor 61 described above. Heart sensors 63 may also include a second electrode (not shown) placed on the skin of the subject 30, such as for example on the wrist of the subject. In embodiments, the heart sensor 63 is configured to continuously monitor and provide the sensor data representative of heart parameters.

An electromyography (EMG) sensor 65 is included in the embodiments shown in FIG. 2A. Electromyography sensor 65 may be any commercially available or otherwise known device that provides sensor data representative of electrical signals of the muscles of the subject 30. The sensor data provided by electromyography sensor 65 may, for example, represent contraction and relaxation of the muscles of the subject 30. Sensor data of this type may, for example, represent pinching forces of a hand of the subject 30, and may be representative of a load being carried by the subject. The electromyography sensor 65 may be used with a band 66, which may for example have features and functionality of the band 62 of the light sensor 61 described above.

A global positioning sensor (GPS) 67 is included in the embodiments shown in FIG. 2A. Global positioning sensor 67 may be any commercially available or otherwise known device that provides sensor data representative of the position and optionally orientation of the subject 30, or portions of the body of the subject to which the sensor is attached. Global positioning sensor 67 may, for example, be configured to operate in connection with one or more satellites. FIG. 7, for example, illustrates exemplary sensor data that can be provided by a global positioning sensor 67. When positioned at a location corresponding to a core of the body of the subject 30, such as for example adjacent a bottom portion of the shirt 16 as shown in FIG. 2A, the sensor data provided by the global positioning sensor 67 may be used as a reference with respect to other sensors, for example without introducing noise of limb movement.

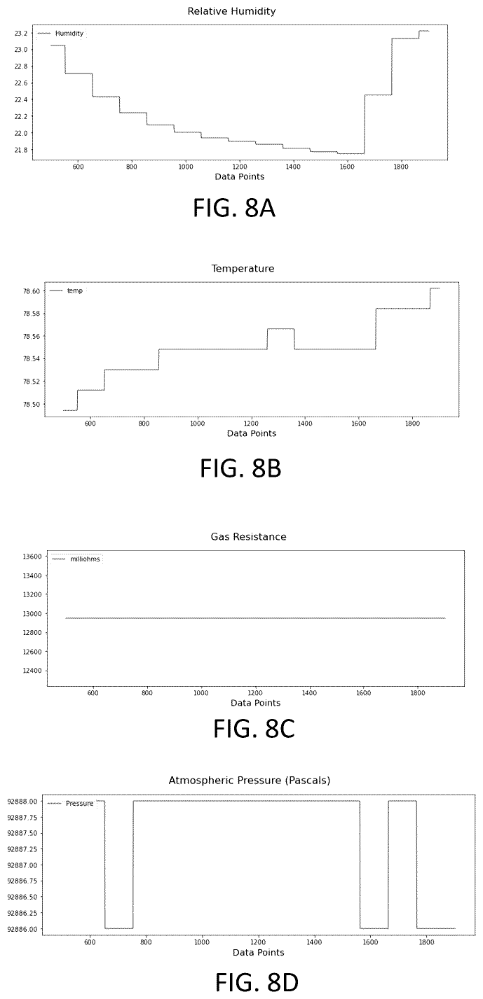

An environmental sensor 68 is included in the embodiments shown in FIG. 2A. Environmental sensor 68 may be any commercially available or otherwise known device that provides sensor data or information representative of the environment in which the subject 30 is located. For example, the environmental sensor 68 may provide sensor data representative of temperature, barometric pressure, gasses, altitude, humidity levels and levels of particulates in the air. FIGS. 8A-8D, for example, illustrate exemplary sensor data from an environmental sensor 68 in the form of relative humidity, temperature, gas resistance and atmospheric pressure, respectively. In the illustrated embodiments, the environmental sensor 68 is at a position corresponding to the chest zone 36, and may provide sensor data representative of humidity and/or pressure or other environmental changes associated with breathing events of the subject 30.

A bend sensor 69 is included in the embodiments shown in FIG. 2A. Bend sensor 69 may be any commercially available or otherwise known device that provides sensor data or information representative of a change in angle between its opposite end portions. In embodiments, the bend sensor 69 may be at a position corresponding to the midline 48. The illustrated embodiments, for example, show the bend sensor 69 at a position corresponding to the upper abdomen zone 38, where measured sensor data may represent changes in the angle of the upper abdomen of the subject 30 during events such as breathing events. Although bending and angles of the subject 30 may be measured or determined from sensor data provided by other sensors, such as for example motion sensors 20a and/or 20b, it may be desirable to additionally and/or alternatively, use sensor data directly representative of bend angles from bend sensor 69. Embodiments may also include bend sensors such as 69 at other positions, such as for example in the lower abdomen zone 40. Coughs and other breathing events of the subject 30 may be initiated in the lower abdomen zone 40 of the subject 30, and can be detected by a bend sensor 69 as well as by motion sensors such as 20a and/or 20b. Although shown generally as being oriented along a vertical, longitudinal (e.g., superior-inferior) axis, changes in bend angles of portions of the body of the subject 30 may also be measured by bend sensors 69 at offset positions and angled orientations.

A stretch sensor 70 is included in the embodiments shown in FIG. 2A. Stretch sensor 70 may be any commercially available or otherwise known device that provides sensor data representative of a change in length, such as for example by measuring changes in resistance and capacitance. Stretch sensors 70 may also be capable of measuring changes in contact pressure, in embodiments. Stretch sensor 70 is shown generally as being oriented along a transverse (e.g., medial-lateral) axis in upper abdomen zone 38, but may be mounted at offset positions, angled orientations and/or at other locations in other embodiments. For example, the stretch sensor 70 may be at a position that provides optimized sensor data in connection with breathing events, and can indicate changes in the shape of the body of the subject 30. Sensor data provided by the stretch sensor 70 may, for example, represent stretching of the shirt 16 during breathing and other events of the subject 30, and may be represented by length changes via a two spring model with the material of shirt 16 being the first spring and the stretch sensor 70 being the second sensor.

Although shown at particular locations on the shirt 16 and body of the subject 30, sensors 18 are positioned at other locations of the shirt or body of the subject in other embodiments. For example, the locations of the sensors 18 may be determined based on factors such as optimization of signal strength provided, relevance of sensor data to the events, such as breathing events desired to be identified and classified, and comfort and fit with respect to the shirt 16. Although one sensor of many of the various different types, or more one in the case of motion sensors 20, are shown for purposes of example, other embodiments include more or fewer sensors 18. By way of example, sensors 18 may be incorporated onto the shirt 16 by approaches and structures such as pockets, adhesive, sewing and/or hook and loop fasteners. In embodiments, for example, the sensors 18 can be incorporated into a sensor harness such as that described in co-pending U.S. provisional application No. 63/442,886 filed on Feb. 2, 2023, and entitled Electronics Harness for Smart Garments, or co-pending U.S. application Ser. No. 17/940,507, filed on Sep. 8, 2022, and entitled Garment including Electronic Devices, both of which are incorporated herein by reference in their entirety and for all purposes.

In embodiments, one or more, or all of the sensors 18 are wireless devices configured to communicate their associated sensor data to the data transfer device 22 on the shirt 16 via the communication channels 24. Additionally, or alternatively, the data transfer device 22 may be in close proximity to the sensors 18, such as for example a mobile phone or device. In embodiments, one or more, or all of the sensors 18 are wireless devices configured to communication the associated sensor data directly to the computer system 14 via the communication channels 24. In embodiments of smart garment 12, the data transfer device 22 may include an electronic component configured to be coupled to one or more, or all of the sensors 18, for example by a releasable connector plug (not shown). Such an electronic component may be configured to be coupled to the sensors 18 so as to facilitate electrical and mechanical connection of the electronic component and the disconnection of the electronic component from the sensors 18. The electronic component may for example include a wireless transmitter to transmit sensor data from the sensors 18 to the computer system 14 via the network 26. Alternatively, or additionally, the electronic component may include electronic memory that stores the sensor data from the sensors 18, for download or other transfer to the computer system 14. Exemplary connector plugs and electronic components of these types are disclosed, for example, in the above identified U.S. provisional application No. 63/442,886 that is incorporated herein.

Data transfer device 22 may also transfer data from the computer system 14 to one or more, or all of the sensors 18 in embodiments. In embodiments, the data transfer device 22 may include an electronics module comprising processor, battery, antenna and/or memory, and may be configured to provide all or portions of the processing functionality of the computer system 14 (e.g., to perform the methods 100 and 300 described below). The electronic components may be housed in an enclosure that is waterproof, and releasably attached to the shirt 16 by, for example, one or more pockets, hook and loop patches, adhesive or fasteners. The above-identified U.S. provisional application No. 63/442,886 that is incorporated herein, for example, discloses structures and approaches, including pockets on waistbands, for releasably retaining data transfer devices such as 22 on the shirt 16. Advantages of releasably retaining all or portions of the data transfer device 22 on the shirt 16 include wash isolation and reuse of the shirt.

Although sensors 18 are described above as being mounted to, located on or otherwise configured for use in connection with the shirt 16, other embodiments of system 10 include sensors that are configured for use with other structures. For example, auxiliary sensors may be mounted to or located on a removable wrist band or wristwatch, or socks, pants or hat worn by or positioned on the subject 30 (not shown).

Embodiments of computer system 14 use one or more models to identify breathing events associated with sensor data provided by the sensors 18 of the subject 30 wearing the smart garment 12, and to classify the identified breathing events as representing one or more event types such as particular types of breathing events. In some examples, one or more of the models are machine learning models. In some examples, one or more of the models are artificial neural networks. Alternatively, or additionally, in some examples, one or more of the models are statistical models. The models are effectively digital representations of the breathing events based upon sensor values that correspond to or characterize the breathing events. Sensor data received from the sensors 18 is applied to and/or compared to the models to identify and classify the sensor data as being associated with one or more breathing events. In embodiments, for example, computer system 14 includes models characterizing breathing events and associated breathing event features for (1) a normal intensity breathing event, (2) a medium intensity breathing event, (3) a high intensity breathing event, (4) a low intensity talking event, (5) a medium intensity talking event, (6) a high intensity talking event, (7) a cough event, (8) a sneeze event, (9) a choke event, (10) a scream event, and/or (11) an apnea event.

In embodiments, computer system 14 uses breathing event features in connection with the models to identify and classify the breathing events. Breathing event features are portions of the sensor data from one or more of the sensors 18 that effectively define particular types of breathing events. For example, the magnitudes or intensities of sensor data may define different breathing events. The relative timing of activity defined by the sensor data, either alone (e.g., in from one sensor), or in combination with activity or lack of activity defined by other sensors, may be define different breathing events. In embodiments, one or more of the breathing event features may be defined by the activities of a plurality of the sensors 18.

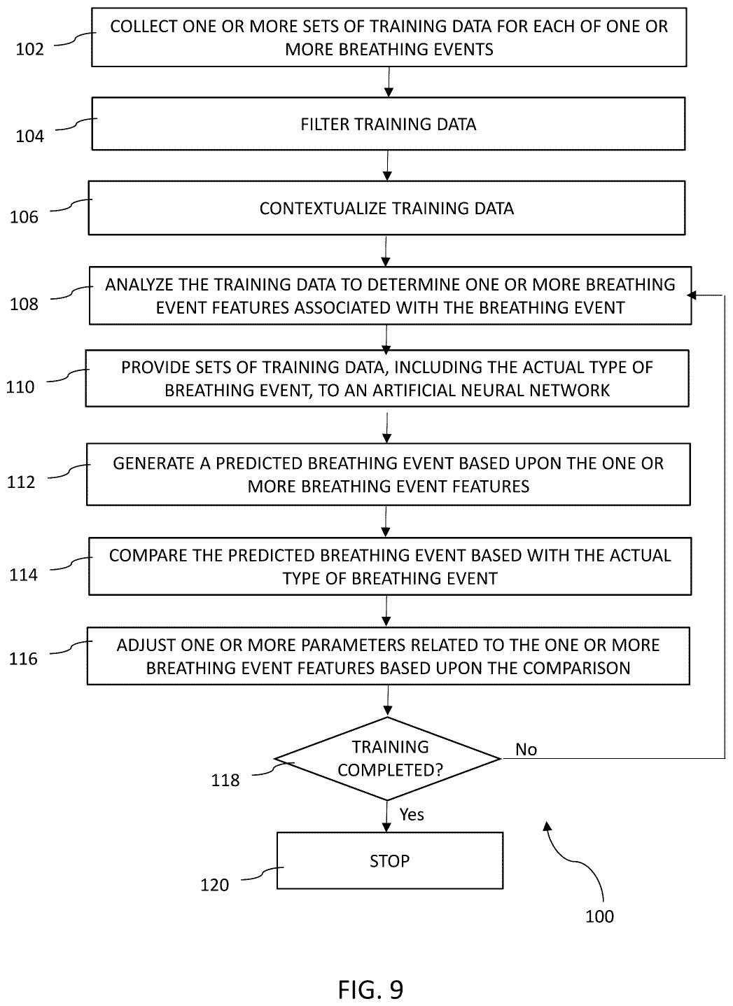

FIG. 9 is a diagrammatic illustration of a method 100 for training a machine learning model, to identify breathing events. The model uses breathing event features to classify and describe certain breathing events in accordance with embodiments. As shown, the method 100 includes process 102 for collecting sets of calibration or training data, process 104 to filter the sets of training data, process 106 for contextualizing the sets of training data, process 108 for analyzing the sets of training data to determine breathing event features, process 110 for providing the sets of training data and features to an machine learning model for training, process 112 for generating predicted breathing events, process 114 for comparing the predicted breathing events with the stored, actual, and/or labeled breathing event associated with the training data, process 116 for adjusting parameters of the machine learning model, and process 118 for determining whether training of the machine learning model has been completed. Although FIG. 9 illustrates a selected group of processes for the method 100, there can be many alternatives, modifications and variations. For example, some of the processes may be expanded and/or combined. Other processes may be inserted. Some of the processes may be optional and need not be performed. Depending upon the embodiment, the sequence of the processes may be interchanged with others replaced. For example, some or all of the processes 102, 104, 106, 108, 110, 112, 114, 116 and 118 are performed by a computing device or a processor directed by instructions stored in memory. As an example, some or all processes of the method 100 are performed according to instructions stored in a non-transitory computer-readable medium. It should be appreciated that while the process 108 and 110 are in the order and describe training classical supervised machine learning model types, such as KNN or decision trees, the order may be switched for an artificial neural network or deep learning approach. While process 108 determines breathing event features, such as breath angle or sensor velocity as variables in classical models, in the event of a convolutional neural network, the contextual data may be directly presented to the classifier for it to determine features such as hidden or pooling layers.

At the process 102, one or more sets of training data for each of the one or more breathing events is collected according to some embodiments. For example, each of the sets of training data includes sensor data relating to breathing event features of the associated breathing events, and knowledge of the types of those breathing events (e.g., the training data sets are labeled with the associated type of breathing event). The one or more sets of training data can be stored in memory element as stored breathing event data for later comparison with a breathing event of a subject.

The training data collected at process 102 may be sensor data from the sensors 18 when the sensors are at one or more known states or upper body poses or positions corresponding to the breathing events. For example, the known states or upper body poses may be static states corresponding to predetermined positions and associated physiologic data of the body of the subject 30 wearing the shirt 16 during the breathing events. Alternatively, or additionally, the known states may be dynamic states corresponding to movement of the body of the subject 30 and associated physiologic data during the associated breathing events.

FIG. 2A, for example, shows the shirt 16 being worn by the subject 30 in a neutral position or pose, where the upper body, including the torso 32, is in a typical upright position (e.g., when the subject is standing or seated), with both their left and right arms hanging or otherwise extending downwardly along the sides of the torso. Sensor data collected from the sensors 18 when the subject 30 is at the position shown in FIG. 2A may be considered baseline position sensor data, and may be used for calibration or reference. In embodiments, training data may also be collected from the sensors 18 when the subject 30 is in other static positions or poses, such as for example with their left and right arms extending straight outwardly from their torso 32 (e.g., in a “T” position). At process 102, training data can also be collected from the sensors 18 when the subject 30 is at a plurality of static positions or poses for each of the breathing events that the system 10 is configured to identify and classify. For example, for each breathing event, the calibration data can be collected from the sensors 18 when the subject is at two or more of a sequence or series of positions or poses they typically have during the associated breathing events (e.g., the breathing event is represented by a sequence of static poses corresponding to poses of an actual breathing event.

The training data collected during the process 102 may be used by the computer system 14 to characterize or “understand” the orientation of sensors 18 with respect to unique geometry of the subject 30 or other subject providing the training or calibration information. When used for calibration purposes, for example, the training data may, be used to adjust or compensate for particular or unique fits of the shirt 16 on the subject 30. For applications where the absolute position of the subject 30 is used, the subject may orientate during the process 102 in a known direction, such as for example north. Accuracy of the training data generated during step 102 may be enhanced, for example to compensate for drift in the sensors 18, if the subject 30 periodically recalculates to the known orientation.

By way of example, training data can be collected from one or more of the motion sensors 20, including motion sensors 20a and 20b, when the subject 30 is at a first stage such as the completion of a breathing event, at the time of a maximum exhale and when the lungs are relatively empty. Training data can be collected from one or more of the motion sensors 20, including motion sensors 20a and 20b, during a subsequent stage of the breathing event, such as at the time of a maximum inhale and when the lungs are relatively full. Sensor data from the sensors 18, including motion data from the motion sensors 20, may differ, for example in magnitude, for each of the breathing events. For example, normal, medium and high intensity breathing events, and low intensity, normal intensity, medium intensity and high intensity talking events may be distinguishable by different motion data from motion sensors 20. As another example, motion data from the motion sensors 20 can be collected when the subject is instructed to perform each of one or more of the different types of breathing events.

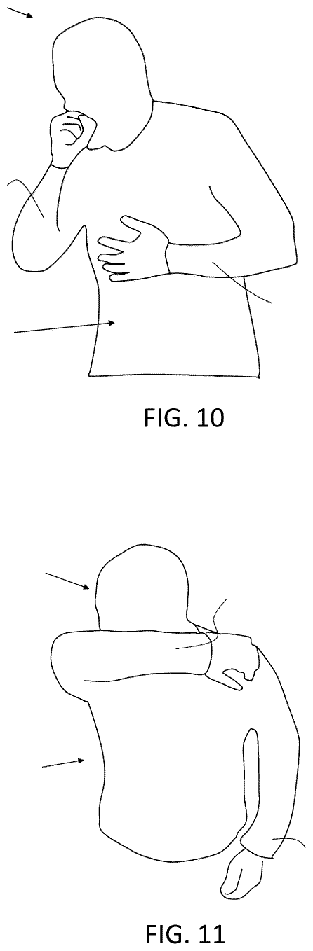

FIG. 10 illustrates the subject 30 during a high intensity breathing event pose. The left arm of the subject 30 is bent upwardly with the hand covering the mouth. The head is angled downwardly. Training data can be collected from the sensors 18 when the subject is in the pose shown in FIG. 10, where for example different portions of the subject's torso 32 such as the chest zone 36, upper abdomen zone 38 and lower abdomen zone 40, and the arm zones 51 and 53, are at different positions with respect to their baseline positions, and can be used to characterize a high intensity breathing event and to distinguish the high intensity breathing event from other breathing events.

FIG. 11 illustrates the subject 30 during a medium intensity breathing event pose. The subject 30 is shown with their right arm upright and bent such that the subject is breathing into the right arm. The head of the subject is angled downwardly. The left arm of the subject 30 remains in the neutral position. Training data collected from the sensors 18 when the subject 30 is in the pose shown in FIG. 11, where for example different portions of the subject's torso 32 such as the chest zone 36, upper abdomen zone 38 and lower abdomen zone 40, and arm zones 51 and 53, are at different positions with respect to their baseline positions, and at different positions with respect to their positions in the high intensity breathing event pose shown in FIG. 10, can be used to characterize a medium intensity breathing event and to distinguish the medium intensity breathing event from other breathing events.

Calibration and/or training data may be collected from sensors 18 at process 102 when the sensors are at dynamic states. For example, a subject 30 wearing the shirt 16 can move through a range of motion corresponding to any one or more of the breathing events that the computer system 14 is configured to classify, and the sensor data collected from the sensors 18 during that range of motion can be used to characterize the breathing event. Training data for any or all of the breathing events the computer system 14 is configured to identify and classify can be collected in the manners described above by process 102.

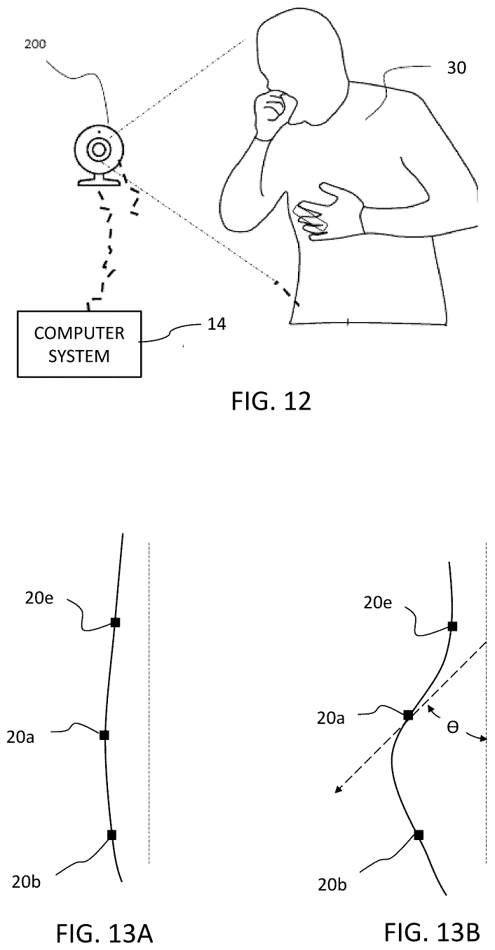

FIG. 12 illustrates embodiments of another approach by which training data can be collected by process 102. The illustrated embodiment includes a video camera system 200 that collects video images of the subject 30 wearing the smart garment 12 while the subject takes static and dynamic poses of various breathing events such as those described above. In addition to the sensor data received from the sensors 18, data from the video images can be processed by the computer system 14 to determine information such as positions of the torso zone 34 and/or arm zones 51, 55 angles between various zones such as between upper abdomen zone 38 and lower abdomen zone 40, and joint angles (e.g., between the upper and lower arms), and velocities. Comparisons between the data and information obtained by the camera system 200 and sensor data from the sensors 18 can be used for calibration and training purposes.

Referring back to FIG. 9, at the process 104, all or portions of the training data may be filtered. Conventional or otherwise known filtering approaches can be performed at process 104. As examples, outlying data can be removed, and/or smoothing functions can be applied (e.g., to dynamic state data). Other non-limiting examples of filtering that may be performed at process 104 include noise removal and/or band pass filtering. Process 104 is optional, and in embodiments only some of the training data, such as for example certain types, or none of the training data, is filtered at process 104.

At process 106, all or portions of the training data may be contextualized or converted into context data. Contextualizing data, also known as context data, at process 106 may include converting all or portions of the training data into other forms that may be useful in connection with the method 100. As an example, the training data may be converted to more human relatable values at process 106. As another example, training data in the form of acceleration data, or acceleration data and gyroscope data, may be converted into joint angles by approaches such as Kalman calculations. Process 106 is optional, and in embodiments only some of the training data, such as for example certain types, or none of the training data, is contextualized at process 106.

FIGS. 13A and 13B, illustrate an exemplary process 106 by which an angle θ can be determined from motion data from motion sensors 20a, 20b and 20e. Through processing of acceleration and gyroscope data from sensos 20a, 20b and 20e, angle θ may be computed or derived. FIG. 13A represents a first spline fit between the positions of sensors 20a, 20b and 20e to define a breath profile for a neutral position or low intensity breathing event. FIG. 13B represents a second spline fit to the positions of sensors 20a, 20b and 20e to define a breath profile for a breathing event having a higher intensity than that of the breathing event shown in FIG. 13A. As yet other examples, any combination of sensor data from motion sensors 20 may define arm movements of the subject 30 during the different breathing events. In still other embodiments, process 106 can process sensor data from any one or more of sensors 18 to determine biological and more human relatable data in the context of a particular application of the smart garment 12. For example, in connection with shirt 16, the body of the subject 30 may have known limitations, such as the elbow bending only, and not rotating. Process 106 may therefore determine probabilities of its human relatable values of motion and alert the user of potential errors of the need to recalibrate the system 10.

At the process 108, the contextualized training data is evaluated to determine one or more breathing event features associated with the breathing event. By way of example, the magnitude of the angle θ of FIG. 13A may be a breathing event feature for use with a classical or deep learning machine learning model. In addition, the rate of change of angle θ may be a breathing event feature. Furthermore, the maximum value of angle θ over a time period may be a breathing event feature. Process 108 yields variables likely to predict classifications of breathing events by the one or more machine learning models. According to certain embodiments, the one or more breathing event features indicate or define the type of the breathing event. For example, identified breathing event features associated with the motion sensors 20, such as for example the motion sensors 20a, 20b and 20e, may represent (1) a normal intensity breathing event, (2) a medium intensity breathing event, (3) a high intensity breathing event, (4) a low intensity talking event, (5) a medium intensity talking event, (6) a high intensity talking event, (7) a cough event, (8) a sneeze event, (9) a choke event, (10) a scream event, and/or (11) an apnea event. Identified breathing event features associated with other sensors 18, alone or in combination with those associated with the motion sensors 20, for breathing events of these types may also be identified at the process 110.

At the process 110, one or more of the sets of training data are provided to train the machine learning model. In examples where the training data was filtered by process 104 and/or contextualized by process 106, the filtered and/or contextualized training data may be provided to the machine learning model at process 108. Labels or other information identifying the type of breathing event associated with the training data, is also provided to the machine learning at process 108. As an example, the machine learning model may be a decision tree network, logistic regression classifier, an artificial neural network, a convolutional neural network, a recurrent neural network, a modular neural network, or any other suitable type of machine learning model.

At the process 110, the training data of each set is analyzed by the machine learning model to determine one or more breathing event features associated with the breathing event.

At the process 112, a predicted breathing event is generated by the machine learning model based upon the breathing event features identified at process 110. For example, in generating the predicted breathing event, one or more parameters related to the one or more breathing event features are calculated by the machine learning model (e.g., weight values associated with various layers of connections.). In connection with the embodiments described above, the predicted breathing event generated at the process 112 may be one of (1) a normal intensity breathing event, (2) a medium intensity breathing event, (3) a high intensity breathing event, (4) a low intensity talking event, (5) a medium intensity talking event, (6) a high intensity talking event, (7) a cough event, (8) a sneeze event, (9) a choke event, (10) a scream event, and/or (11) an apnea event. Alternatively, an undetermined type of breathing event, or no breathing event, may be the predicted breathing event generated at the process 112.

At the process 114, the predicted breathing event is compared to the actual type or breathing event to determine an accuracy of the predicted breathing event. By some embodiments, the accuracy is determined by using a loss function or a cost function of the set of training data.

At the process 116, based upon the comparison, the one or more parameters related to the breathing event features are adjusted by the machine learning model. For example, the one or more parameters may be adjusted to reduce (e.g., minimize) the loss function of the cost function.

At the process 118, a determination is made on whether the training has been completed. For example, training for one set of training data may be completed when the loss function or the cost function for the set of training data is sufficiently reduced (e.g., minimized). As an example, training for the machine learning model is completed when training yields acceptable accuracy between predicted and known labels for one or more datasets. In embodiments, if the process 118 determines that training of the machine learning model is not yet completed, then the method 100 returns to the process 108 in an iterative manner until the training is determined to be completed. In embodiments, if the process 118 determines that training of the machine learning model is completed, then the method 100 stops as indicated at process 120. The trained machine learning model effectively possesses existing knowledge of which breathing event features are desirable of useful in terms of identifying and classifying breathing events.

Although the method 100 described above trained one machine learning model for purposes of identifying a plurality of different types of breathing events, in other embodiments each of one or more machine learning models may be trained for purposes of identifying one, or more than one but less than all, of the plurality of different types of beathing events. Stated differently, machine learning models may be trained by the method 100 for purposes of identifying and classifying one, or more than one, type of breathing events.

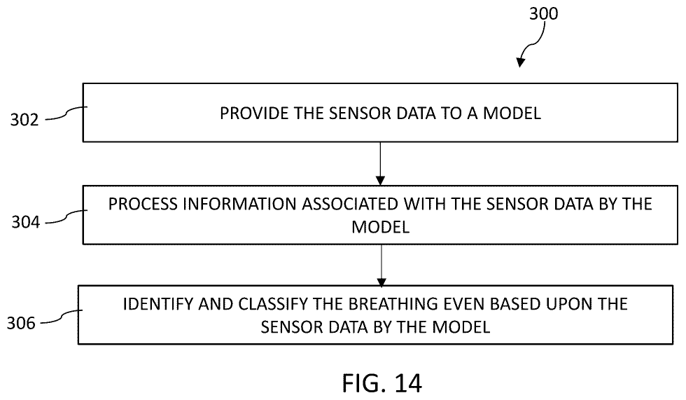

FIG. 14 is a diagrammatic illustration of an exemplary method 300 for identifying and classifying, or determining breathing events, in accordance with embodiments. As shown, the method 300 includes process 302 for providing the sensor data to one or more models, process 304 for processing the sensor data by the model, and process 306 for determining or identifying a breathing event by the one or more models. In some embodiments, the model is a statistical model. Alternatively, or additionally, one or more, or all of the models may be a machine learning model, such as for example an artificial neural network trained in accordance with the method 100 described above in connection with FIG. 9. Although method 300 is described as using a selected group of processes, there can be many alternatives, modifications, and variations. For example, some of the processes may be expanded and/or combined. Other processes may be inserted. Depending upon the embodiment, the sequence of processes may be interchanged with others replaced. For example, some or all processes of the method 300 are performed by a computing device or a processor directed by instructions stored in memory. As an example, some or all processes of the method are performed according to instructions stored in a non-transitory computer-readable medium.

At the process 302, the sensor data from the garment 12 is provided to the model. For example, the sensor data are collected from the sensors 18, including the motion sensors 20. In some embodiments, the sensor data includes at least sensor data from the motion sensors 20a and 20b.

At the process 304, the sensor data is processed similar to processes 104, 106 and 108, in that process 304 filters, contextualizes and generates features, from the sensor data.

At the process 306, the model determines or identifies and classifies breathing events based upon the processed sensor data. For example, in embodiments the model identifies or classifies the sensor data as being representative of one of at least one of (1) a normal intensity breathing event, (2) a medium intensity breathing event, (3) a high intensity breathing event, (4) a low intensity talking event, (5) a medium intensity talking event, (6) a high intensity talking event, (7) a cough event, (8) a sneeze event, (9) a choke event, (10) a scream event, and/or (11) an apnea event.

Other embodiments use other approaches for identifying and classifying breathing events based upon the sensor data. For example, during calibration operations of the types described above breathing event features or other breathing event data associated with the different types of breathing events can be identified (e.g., by trained machine models or human labeling), and stored in association with the breathing events. In effect, each of the different types of breathing events can be defined, characterized or represented by a set of one or more breathing event features that are unique to the breathing events. Sensor data received from the sensors 18 on the smart garment 12, including sensor data from motion sensors 20 such as motion sensors 20a and 20b, may then be compared to the stored breathing event features to identify and classify the breathing events represented by the sensor data. Feature extraction, pattern recognition and other processing methodologies may be used in connection with such approaches for identifying and classifying breathing events.

It will be appreciated that system 10 may be used to characterize or classify different breathing events. As one embodiment, system 10 may be utilized by a pharmaceutical company wanting to improve the lives of people with a breathing condition such as but not limited to chronic obstructive pulmonary disease (COPD), emphysema, Covid, asthma or lung cancer. The pharmaceutical company may first test patients utilizing system 10 to better understand the disease and type of coughs associated with it. Types of coughs may range from just a mild clearing of a throat to an intense cough having both arms covering one's mouth. Machine learning models utilizing features such as, maximum value of angle θ over a cough duration, the difference in acceleration between sensors 20C and 20D, maximum and minimum angles of sensors 20k and 20j may be used to predict cough type and severity for a particular breathing disease. The pharmaceutical company may then create new features, or combine features to create novel biomarkers that can indicate disease progression. The pharmaceutical company may then utilize the novel biomarkers to indicate safety and efficacy of a new drug. Ultimately, the pharmaceutical company may use system 10 to remotely monitor patients taking their new drug.

While the embodiment above describes a use for system 10 for people with a disease having a breathing event as a primary condition, system 10 may also be used for people with diseases having breathing events as a secondary condition. For example, seizures can be characterized by motion and breathing events. A first seizure type may be an impairment type wherein a person loses some movement of arms but may still be able to talk. A more intense seizure may be immobility of arms and involuntary breathing events such as talking, crying, or screaming. An even more intense seizure may have involuntary muscle contractions and the absence of breathing (apnea). As a non-limiting example, features of combined average acceleration of sensors 20c and 20d, frequency of accelerations of 20k and 20j and a rolling three second increase in heart rate from sensor 60 may be used to classify a type of seizure. A doctor may have a subject wear system 10 in their normal day to capture data from a potential seizure. System 10 may help the doctor make a diagnosis based upon data including classification of a breathing event.

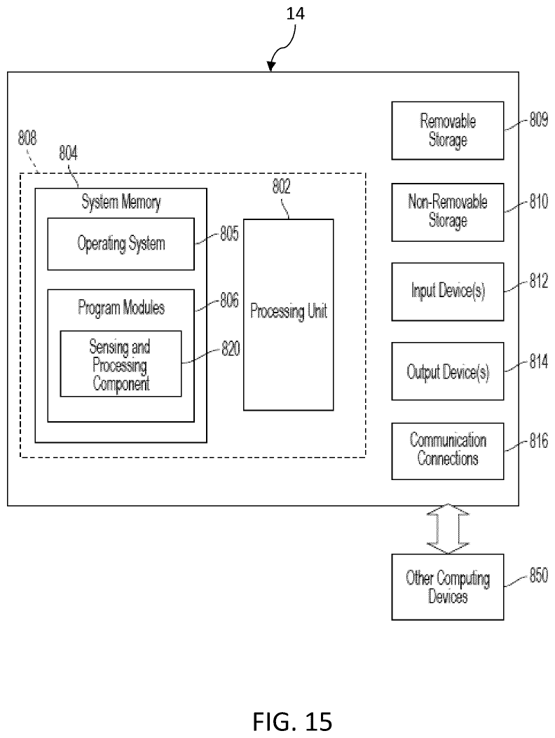

FIG. 15 is a block diagram illustrating physical components (e.g., hardware) of an exemplary computer system 14, in accordance with embodiments. In a basic configuration, the computer system 14 may include at least one processing unit 802 and a system memory 804. Depending on the configuration and type of computer system 14, the system memory 804 may comprise, but is not limited to, volatile storage (e.g., random access memory), non-volatile storage (e.g., read-only memory), flash memory, or any combination of such memories. The system memory 804 may include an operating system 805 and one or more program modules 806 such as a sensing and processing component 820.

The operating system 805, for example, may be suitable for controlling the operation of the computer system 14. Furthermore, embodiments of the disclosure may be practiced in conjunction with a graphics library, other operating systems, or any other application program and is not limited to any particular application or system. This basic configuration is illustrated in FIG. 15 by those components within a dashed line 808. The computer system 14 may have additional features or functionality. For example, the computer system 14 may also include additional data storage devices (removable and/or non-removable). Such additional storage is illustrated in FIG. 15 by a removable storage device 809 and a non-removable storage device 810.

As stated above, a number of program modules and data files may be stored in the system memory 804. While executing on the processing unit 802, the program modules 806 (e.g., the sensing and processing component 820) may perform processes including, but not limited to, the aspects, as described herein, e.g., the processing of the sensor data from sensors 18 and the methods 100 and 300.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. For example, embodiments of the disclosure may be practiced via a system-on-a-chip (SOC) where each or many of the components illustrated in FIG. 15 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the computer system 14 on the single integrated circuit (chip). Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

The computer system 14 may also have one or more input device(s) 812 such as visual image sensors, audio sensors, a sound or voice input device, a touch or swipe input device, etc. The output device(s) 814 such as a display, speakers, etc. may also be included. The aforementioned devices are examples and others may be used. The computer system 14 may include one or more communication connections 816 allowing communications with other computing devices 850 (e.g., computing devices 128 and/or 130). Examples of suitable communication connections 816 include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 804, the removable storage device 809, and the non-removable storage device 810 are all computer storage media examples (e.g., memory storage). Computer storage media may include RAM, ROM, electrically erasable programmable read-only memory (EEPROM), flash memory or other memory technology, optical storage, magnetic storage devices, or any other article of manufacture which can be used to store information, and which can be accessed by the computer system 14. Any such computer storage media may be part of the computer system 14. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A method for operating a computing system including one or more processors to identify breathing events of a subject, comprising:
receiving, by the one or more processors, data from a plurality of sensors, including a plurality of motion sensors, mounted to an article positioned on an upper body of the subject, wherein the data includes data associated with movement of an upper body of the subject;
processing the data, by the one or more processors, to identify a breathing event of the subject, wherein the identified breathing event is at least one of a normal intensity breathing event, a medium intensity breathing event, a high intensity breathing event, a low intensity talking event, a normal intensity talking event, a medium intensity talking event, a high intensity talking event, a cough event, a sneeze event, a choke event, a scream event, and an apnea event;
wherein at least two of the plurality of motion sensors are mounted to the article at positions corresponding to a midline of an anterior portion of the subject; and
wherein at least a first of the plurality of motion sensors is mounted to the article at a position corresponding to a position proximate to and above an umbilicus of the subject, and at least a second of the plurality of motion sensors is mounted to the article at a position corresponding to a position proximate to and below the umbilicus of the subject.

2. The method of claim 1, wherein processing the data to identify a breathing event comprises comparing the data to stored breathing event data.

3. The method of claim 2, further comprising receiving and storing the breathing event data.

4. The method of claim 3, wherein the stored breathing event data is representative of one or more upper body poses.

5. The method of claim 4, wherein the stored breathing event data includes data received during a static upper body pose.

6. The method of claim 5, wherein the stored breathing event data includes data received during a dynamic upper body pose.

7. The method of claim 1, wherein processing the data comprises processing the data by a trained model.

8. The method of claim 7, wherein processing the data comprises processing the data by a trained artificial neural network.

9. The method of claim 7, further comprising training the model.

10. The method of claim 1, wherein processing the data comprises:
converting at least portions of the data into context data associated with human-relatable values; and
processing the context data.

11. The method of claim 1, wherein:
receiving the data includes receiving data from each of at least two zones of the article; and
processing the data includes processing the data from the at least two zones.

12. The method of claim 1, wherein receiving the data comprises receiving the data from the plurality of sensors mounted to a shirt.

13. The method of claim 1, wherein processing the data includes identifying features associated with movement of one or more of the subject's chest, upper abdomen, lower abdomen, left arm or right arm.

14. The method of claim 1, wherein processing the data includes identifying breathing event features.

15. The method of claim 1, wherein processing the data to identify a breathing event includes determining an angle of the upper body of the subject based upon data from sensors including the at least two of the plurality of motion sensors mounted at positions corresponding to the midline of the anterior portion of the subject, the at least first of the plurality of motion sensors mounted at the position proximate to and above the umbilicus, and the at least second of the plurality of motion sensors mounted at the position proximate to and below the umbilicus.

16. A system for identifying breathing events of a subject, comprising:
an article configured to be positioned on an upper body of the subject, the article including a plurality of sensors, including a plurality of motion sensors, for sensing subject data; and
a computer system including one or more processors, wherein the computer system is configured to receive the subject data and to identify a breathing event of the subject, wherein the breathing event is at least one of a normal intensity breathing event, a medium intensity breathing event, a high intensity breathing event, a cough event, a sneeze event, a choke event, a low intensity talking event, a normal intensity talking event, a medium intensity talking event, a high intensity talking event, a scream event, and an apnea event;
wherein at least two of the plurality of motion sensors are located on the article at positions corresponding to a midline of an anterior portion of the subject; and
wherein at least a first of the plurality of motion sensors is located on the article at a position corresponding to a position proximate to and above an umbilicus of the subject, and at least a second of the plurality of motion sensors is located on the article at a position corresponding to a position proximate to and below the umbilicus of the subject.

17. The system of claim 16, wherein the plurality of motion sensors comprises one or more motion sensors in each of at least two zones.

18. The system of claim 17, wherein the article comprises a shirt and the at least two zones include zones from a group including a chest zone, an upper abdomen zone, a lower abdomen zone, a left arm zone and a right arm zone.

19. The system of claim 16, wherein the computer system identifies the breathing event at least in part by determining an angle of the upper body of the subject based upon subject data from sensors including the at least two of the plurality of motion sensors mounted at positions corresponding to the midline of the anterior portion of the subject, the at least first of the plurality of motion sensors mounted at the position proximate to and above the umbilicus, and the at least second of the plurality of motion sensors mounted at the position proximate to and below the umbilicus.

20. The system of claim 16, wherein the computer system identifies the breathing event at least in part by comparing the subject data to stored breathing event data.

21. The system of claim 20, wherein the computer system receives and stores the breathing event data.

22. The system of claim 21, wherein the stored breathing event data is representative of one or more upper body poses.

23. The system of claim 22, wherein the stored breathing event data includes data received during a static upper body pose.

24. The system of claim 23, wherein the stored breathing event data includes data received during a dynamic upper body pose.

25. The system of claim 16, wherein the computer system identifies the breathing event at least in part by processing the subject data by a trained model.

26. The system of claim 16, wherein the computer system identifies the breathing event at least in part by:
converting at least portions of the subject data into context data associated with human-relatable values; and
processing the context data.

27. The system of claim 16, wherein the computer system identifies the breathing event at least in part based upon subject data from each of at least two zones of the article; and processing the data from the at least two zones.

28. An article, comprising:
a shirt configured to be worn on an upper body of a subject;
a plurality of sensors, including a plurality of motion sensors, mounted to the shirt for providing subject data, wherein the plurality of motion sensors are configured and arranged to provide subject data characteristic of sufficient features associated with the upper body of the subject, including at least one feature associated with movement of the upper body of the subject, to determine a subject breathing event, wherein the breathing event is at least one of a normal intensity breathing event, a medium intensity breathing event, a high intensity breathing event, a cough event, a sneeze event, a choke event, a low intensity talking event, normal intensity talking event, a medium intensity talking event, a high intensity talking event, a scream event, and an apnea event;
a data transfer structure on the shirt coupled to the plurality of sensors and configured to facilitate transferring the subject data off of the shirt;
wherein at least two of the plurality of motion sensors are located on the shirt at positions corresponding to a midline of an anterior portion of the subject; and
wherein at least a first of the plurality of motion sensors is located on the shirt at a position corresponding to a position proximate to and above an umbilicus of the subject, and at least a second of the plurality of motion sensors is located on the shirt at a position corresponding to a position proximate to and below the umbilicus of the subject.

29. The article of claim 28, wherein the plurality of motion sensors includes one or more sensors on at least two zones of the shirt.

30. The article of claim 28, wherein:
the shirt includes a chest zone, an upper abdomen zone, a lower abdomen zone, a left arm zone and a right arm zone, and
the plurality of motion sensors includes one or more motion sensors on each of the chest zone, upper abdomen zone, lower abdomen zone, left arm zone and right arm zone.
